(19) United States
(12) Patent Application Publication
(10) Pub. No.: US 2003/0166732 A1
(43) Pub. Date: Sep. 4, 2003

(54) Ambroxol for the treatment of painful conditions in the mouth and pharyngeal cavity

(75) Inventors: Anke Esperester, Mainz (DE); Uwe Pschorn, Mainz (DE); Jean-Michel Vix, Wiesbaden (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/376,349

(22) Filed: Feb. 27, 2003

Related U.S. Application Data
(60) Provisional application No. 60/386,164, filed on Jun. 5, 2002.

(30) Foreign Application Priority Data
Feb. 27, 2002 (DE) DE 102 08 313.4

(51) Int. Cl. A61K 38/47; A61K 31/56; A61K 31/137; A61K 31/4709; A61K 31/135

(52) U.S. Cl. 514/649; 514/171; 424/94.61; 514/308; 514/557; 514/518; 514/643

(57) ABSTRACT

The invention relates to the use of ambroxol and the pharmacologically acceptable salts thereof for preparing a pharmaceutical composition for the treatment of painful conditions in the oral and pharyngeal cavity.

### RELATED APPLICATION

This application claims priority benefit of U.S. provisional application serial No. 60/386,164, filed Jun. 5, 2002.

### BACKGROUND TO THE INVENTION

The invention relates to the use of ambroxol and the pharmacologically acceptable salts thereof for preparing a pharmaceutical composition for the treatment of painful conditions in the oral and pharyngeal cavity.

Painkillers for relieving pain in the oral and pharyngeal cavity often have the drawback of side effects, e.g. in the form of local irritations.

The active substance ambroxol (trans-4-(2-amino-3,5-dibromobenzylamino)-cyclohexanol) is a known expectorant and mucolytic. It is used in oral preparations such as syrups, capsules, tablets, inhalable solutions, drops or suckable pastilles. The aim of the present invention is to prepare a well-tolerated active substance for the treatment of pain in the oral and pharyngeal cavities.

### SUMMARY OF THE INVENTION

The invention relates to pharmaceutical compositions comprising ambroxol, bromhexine or one of the pharmacologically acceptable salts thereof. The invention also relates to pharmaceutical compositions comprising ambroxol or one of the pharmacologically acceptable salts thereof and one or more active substances (e.g., antiseptics, vitamins, corticoids, antiphlogistics, antibiotics, antimycotics, and proteolytic enzymes, lysozyme hydrochloride, dipotassium glycyrrhizinate, ammonium glycyrrhizinate, cetylpyridinium chloride, chlorpheniramine maleate, noscapine, dequalinium chloride, dextromethorphane, phenolphthalinate, potassium guaiacolsulphonate, dl-methylephedrine hydrochloride, chlorhexidine hydrochloride, and potassium cresolsulphonate). The invention also relates to methods comprising administering a pharmaceutical composition of the invention for the treatment of painful conditions in the oral and pharyngeal cavity (e.g., acute sore throat, aphthae, gingivitis, parodontopathies, pressure points caused by prostheses, pain after oro-pharyngeal interventions, lesions on the mucous membrane in the oral and pharyngeal cavity and herpes simplex in the oral and pharyngeal cavity). Various formulations are provided in the invention (e.g., solid, suckable or slowly dissolving formulation, semisolid formulation).

### BRIEF DESCRIPTION OF THE FIGURES

FIG. 1 shows the course, over time, of the average change in pain intensity (PID) for the period before taking the tablet (base line) up to 3 hours after taking the first suckable tablet containing 20 mg of ambroxol hydrochloride and placebo.

FIG. 2 shows the course, over time, of the average change in pain intensity (PID) for the period before taking the tablet (base line) up to 3 hours after taking the first suckable tablet containing 20 mg or 30 mg of ambroxol hydrochloride and placebo.

### DESCRIPTION OF THE INVENTION

Surprisingly, it has been found that, when administered locally in suitable doses or concentrations, ambroxol has a very good pain-relieving effect in the oral and pharyngeal cavity in addition to being very well tolerated.

The invention therefore relates to the use of ambroxol or one of the pharmacologically acceptable salts thereof for preparing a pharmaceutical composition for the treatment of pain in the oral and/or pharyngeal cavity, selected from among acute sore throat, aphthae, gingivitis, parodontopathies, pressure points caused by prostheses, pain after oro-pharyngeal interventions, lesions on the mucous membrane in the oral and pharyngeal cavity and herpes simplex in the oral and pharyngeal cavity, particularly aphthae, gingivitis, parodontopathies, pressure points caused by prostheses, pain after oro-pharyngeal interventions, lesions on the mucous membrane in the oral and pharyngeal cavity and herpes simplex in the oral and pharyngeal cavity, most particularly for the treatment of acute sore throats. By acute sore throats are meant severe sore throats, for example inflamed throats with difficulty swallowing or with burning in the throat.

The invention further relates to a pharmaceutical composition containing ambroxol or one of the pharmacologically acceptable salts thereof and one or more active substances selected from among the antiseptics, vitamins, corticoids, antiinflammatories, virostatics, antibiotics, antimycotics and proteolytic enzymes.

Suitable antiseptics are for example cetylpyridinium-Cl, dequalinium-Cl, chlorhexidine-digluconate, benzalkonium-Cl or ethacridine-lactate.

Suitable vitamins are for example dexpanthenol (pantothenic acid) or ascorbic acid.

Suitable corticoids are for example triamcinolone or prednisolone-acetate.

Suitable antiinflammatories are for example benzydamine-Cl or choline salicylate.

Suitable virostatics are for example acyclovir or idoxuridine.

Suitable antibiotics are for example thyrotricin or bacitracin.

Suitable antimycotics are for example amphotericin B or nystatin.

An example of a suitable proteolytic enzyme is lysozyme.

Suitable ethereal oils are for example peppermint oil, thyme or sage oils.

The invention further relates to a pharmaceutical composition containing ambroxol or one of the pharmacologically acceptable salts thereof and one or more active substances, selected from the group consisting of lysozyme hydrochloride, dipotassium glycyrrhizinate, ammonium glycyrrhizinate, cetylpyridinium chloride, chlorpheniramine maleate, noscapine, dequalinium chloride, dextromethorphan, phenolphthalinate, potassium guaiacolsulphonate, dl-methylephedrine hydrochloride, chlorhexidine hydrochloride, and potassium cresolsulphonate.

Ambroxol is a metabolite of the secretolytic bromhexine. The two active substances represent a very well tolerated combination of active substances which positively influences the dual effect of ambroxol.

The invention therefore also relates to a pharmaceutical composition consisting of ambroxol, bromhexine or the pharmacologically acceptable salts thereof and pharmaceutical excipients, preferably with a ratio of ambroxol to bromhexine in the range from 4:1 to 6:1, more preferably 5:1.

A particularly preferred pharmaceutical composition is one wherein the single dose contains 15 to 50 mg of ambroxol, preferably 20 mg of ambroxol.

The invention further relates to a solid, suckable or slowly dissolving form of a pharmaceutical composition containing ambroxol and one or more active substances selected from among the antiseptics, vitamins, corticoids, antiinflammatories, antibiotics, antimycotics and proteolytic enzymes.

The invention further relates to the use of a pharmaceutical composition as described above for preparing a medicament for the treatment of pain in the oral and/or pharyngeal cavity, selected from among acute sore throat, aphthae, gingivitis, parodontopathies, pressure points caused by prostheses, pain after oro-pharyngeal interventions, lesions on the mucous membrane in the oral and pharyngeal cavity and herpes simplex in the oral and pharyngeal cavity, most preferably for treating acute sore throats.

The invention further relates to the use of a pharmaceutical composition consisting of ambroxol hydrochloride, a flavouring, a lubricant, a matrix material, a sweetening agent and a polyethyleneglycol for preparing a pharmaceutical composition for the treatment of pain in the oral and/or pharyngeal cavity, selected from among acute sore throat, aphthae, gingivitis, parodontopathies, pressure points caused by prostheses, pain after oro-pharyngeal interventions, lesions on the mucous membrane in the oral and pharyngeal cavity and herpes simplex in the oral and pharyngeal cavity, most preferably for treating acute sore throats.

Suitable flavourings may be, for example, peppermint, eucalyptus or lemon, preferably peppermint flavouring.

Suitable matrix materials may be, for example, calcium carbonate, calcium phosphate or sorbitol, preferably sorbitol.

Suitable sweetening agents may be, for example, saccharin, saccharin sodium, cyclamate, glycerol or sugar, preferably saccharin sodium.

Suitable tablet lubricants may be, for example, polyethyleneglycols, preferably Macrogol 6000.

Suitable lubricants may be for example talcum or magnesium stearate, preferably talc.

The invention further relates to the use of a suckable tablet containing ambroxol based on sugar alcohols as the matrix material, characterised in that it contains a pharmaceutically acceptable layered silicate and a polyethyleneglycol, optionally together with other pharmaceutical excipients, taste or flavouring agents to prepare a pharmaceutical composition for treating pain in the oral and/or pharyngeal cavity, selected from among acute sore throat, aphthae, gingivitis, parodontopathies, pressure points caused by prostheses, pain after oro-pharyngeal interventions, lesions on the mucous membrane in the oral and pharyngeal cavity and herpes simplex in the oral and pharyngeal cavity, most particularly for the treatment of acute sore throats.

The invention further relates to the use of ambroxol for preparing a pharmaceutical composition with a pain-relieving effect lasting for a period of at least 3 hours, preferably more than 3 hours, after administration.

The invention also relates to the use of a pharmaceutical composition containing ambroxol for preparing a pharmaceutical composition with a pain-relieving effect lasting for a period of at least 3 hours, preferably more than 3 hours, after administration.

The pharmaceutical composition according to the invention is preferably administered 1 to 6 times, preferably 2 to 4 times a day.

Acids suitable for forming salts of ambroxol include for example hydrochloric acid, hydrobromic acid, sulphuric acid, phosphoric acid, nitric acid, oxalic acid, malonic acid, fumaric acid, maleic acid, tartaric acid, citric acid, ascorbic acid and methanesulphonic acid, preferably hydrochloric acid.

The activity of ambroxol according to the invention is intended to be illustrated by the following examples of clinical trials which investigate the effectiveness of different strengths of suckable tablets containing ambroxol. These are intended solely to illustrate the invention and are not to be regarded as limiting.

### EXAMPLE 1

Investigation of the Activity and Tolerance of Suckable Tablets Containing 20 mg of Ambroxol Hydrochloride (Trans-4-[(2-amino-3,5-dibromo-benzyl)amino]Cyclohexano Hydrochloride, CAS Reg. No. 18683-91-5) Compared With a Placebo in Treating Acute Sore Throats

A multi-centred, prospective, placebo-controlled, randomised double-blind trial was carried out over two days' treatment with up to 6 suckable tablets containing ambroxol hydrochloride per day.

Patients: 218 patients (97 men, 121 women) with an average age of 39.4±15 years (range from 17-81 years) were recruited; of these 215 patients were treated: 107 with 20 mg of ambroxol and 108 with placebo. 26 patients stopped the treatment early (13 in each treatment group). The intent-to-treat (ITT) population consisted of 208 patients (105 treated with ambroxol and 103 given the placebo); 196 patients formed the per-protocol (PP) population (97 with test substance and 99 with placebo). For the drug safety analysis, all the patients treated were studied.

Treatments: Double-blind treatment with up to 6 suckable tablets per day, which either contained 20 mg of ambroxol or constituted a placebo (suckable tablet without the active substance, but with a marked flavour of peppermint similar to the test substance).

End points: The average pain reduction, weighted for time, during the first 3 hours after administration of the first suckable tablet, standardised to the degree of initial pain (SPIDₙₒᵣₘ); moreover, the assessment of effectiveness and tolerance by the. patient at the end of each day of treatment.

Results: In both treatment groups there was a reduction in the intensity of pain; the average SPIDₙₒᵣₘ (±SD) after the first suckable tablet was 0.39±0.27 for 20 mg ambroxol hydrochloride and 0.28±0.25 for placebo.

The superiority of the ambroxol over the placebo was apparent from a statistically significant treatment effect (p=0.0029; 95% confidence interval for the average difference between the ambroxol treatment groups minus placebo: 0.04 to 0.18). At the end of each successive day of ambulant treatment with up to 6 suckable tablets a statistically significantly larger number of patients reported a higher degree of effectiveness for the active treatment with ambroxol hydrochloride than for the administration of the placebo. The test substance was found to be tolerated just as well as the placebo.

Conclusion: The administration of suckable tablets containing 20 mg of ambroxol hydrochloride to patients with acute sore throat has an effective pain-relieving effect which is superior to the inherently beneficial effect of sucking a placebo.

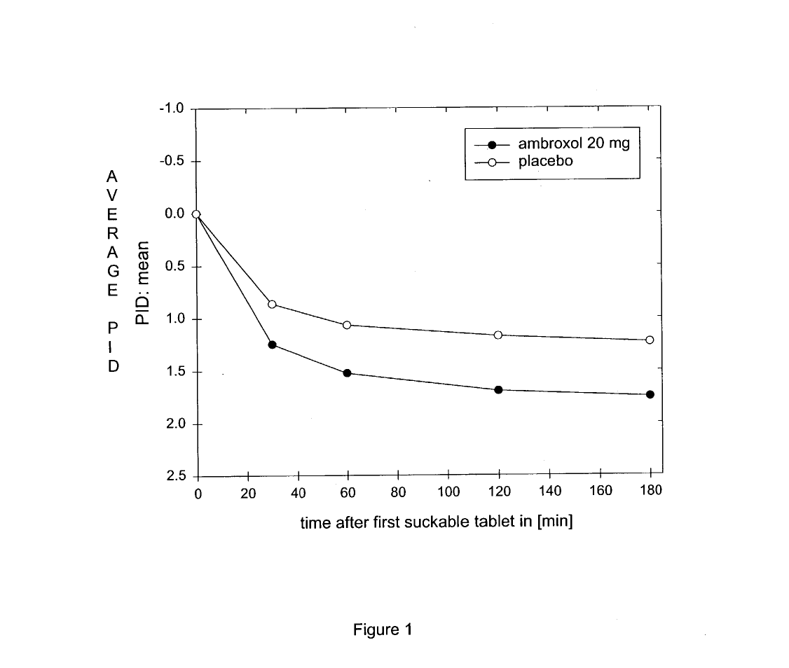

FIG. 1 shows the course, over time, of the average change in pain intensity (PID) for the period before taking the tablet (base line) up to 3 hours after taking the first suckable tablet containing 20 mg of ambroxol hydrochloride and placebo.

### EXAMPLE 2

Investigation of the Activity and Tolerance of Suckable Tablets Containing 20 or 30 mg of Ambroxol Hydrochloride (Trans4-[(2-amino-3,5-dibromo-benzyl)amino]Cyclohexano Hydrochloride, CAS Reg. No. 18683-91-5) Compared With a Placebo in Treating Acute Sore Throats

A multi-centred, prospective, placebo-controlled, randomised double-blind trial was carried out over three days' treatment with up to 6 suckable tablets containing ambroxol hydrochloride per day.

Patients: 331 ambulant patients with acute uncomplicated sore throats of at least moderate severity but with no bacterial pharyngitis were investigated.

Treatments: Double-blind treatment with up to 6 suckable tablets per day containing either 20 mg or 30 mg of ambroxol hydrochloride or constituting a placebo (suckable tablet without the active substance, but again with a marked flavour of peppermint similar to the test substance).

End points: The average pain reduction, weighted for time, during the first 3 hours after administration of the first suckable tablet, standardised to the degree of initial pain (SPIDₙₒᵣₘ); moreover, the assessment of effectiveness and tolerance by the patient at the end of each day of treatment.

Results: All the treatments led to a reduction in the intensity of pain; the average SPIDₙₒᵣₘ (±SD) after the first suckable tablet was taken was 0.53±0.28 or 0.50±0.30 for 20 mg and 30 mg ambroxol hydrochloride, respectively, and 0.38±0.28 for placebo. The effect of the treatment was statistically significant. The superiority of the active treatments over the placebo could be clearly demonstrated (95% confidence interval (Cl) for the average differences between the groups treated with suckable tablets containing 20 or 30 mg of ambroxol minus placebo: 0.08 to 0.23 or 0.05 to 0.20). At the end of each successive day of ambulant treatment with up to 6 suckable tablets per day a statistically significantly larger number of patients reported a higher degree of effectiveness for the active treatments with ambroxol hydrochloride than for the administration of the placebo. The test substance was found to be tolerated just as well as the placebo in all dosages.

Conclusion: The administration of suckable tablets containing 20 or 30 mg of ambroxol hydrochloride to patients with acute sore throat has a markedly effective pain-relieving effect which is superior to the inherently beneficial effect of sucking a placebo. Both doses were tolerated equally well.

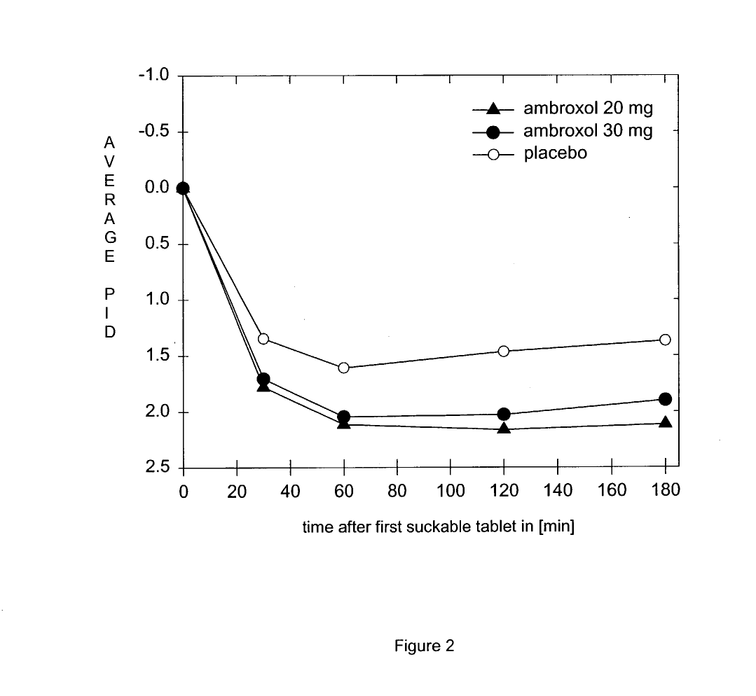

FIG. 2 shows the course, over time, of the average change in pain intensity (PID) for the period before taking the tablet (base line) up to 3 hours after taking the first suckable tablet containing 20 mg or 30 mg of ambroxol hydrochloride and placebo.

Ambroxol may be used on its own or combined with other pharmacologically active substances. It may be applied in any of the preparation forms which are suitable for local use. Preparations suitable for sucking or dissolving slowly in the mouth include, for example, tablets or sweets based on sugar or sugar substitutes or pastille-like products with a gum arabic or gelatine base.

Examples of semisolid preparations for application to the oral mucosa include gels, for example, especially gels based on cellulose or acrylate.

Suitable solutions for spraying, gargling and rinsing include aqueous preparations, advantageously with the addition of viscosity-increasing substances such as modified celluloses, acrylic acid derivatives or polyvinyl compounds.

In addition, the semisolid and liquid forms in particular may contain sweetening agents and moisture retainers such as glycols and sugar alcohols, for example.

All the forms are flavoured in the conventional way, e.g. by the addition of ethereal oils.

The preparations may be produced by methods known in pharmacy.

The following examples of pharmaceutical formulations illustrate the present invention without restricting its scope:

Formulation 1)

| Suckable pastille | per pastille | |
|---|---|---|
| ambroxol hydrochloride | 20.0 | mg |
| peppermint flavouring | 16.0 | mg |
| sorbitol | 1373.5 | mg |
| saccharin sodium | 0.5 | mg |
| Macrogol 6000 | 30 | mg |
| talc | 60 | mg |

Formulation 2)

| Suckable pastille | per tablet | |
|---|---|---|
| Ambroxol hydrochloride | 20.0 | mg |
| Lysozyme hydrochloride | 5.0 | mg |
| Dipotassium glycyrrhizinate | 2.5 | mg |
| Cetylpyridinium Chloride | 1.0 | mg |
| Chlorpheniramine Maleate | 1.0 | mg |
| Xylitol | 920.5 | mg |
| D-Mannitol | 9.5 | mg |
| Polyvinylpyrrolidone | 21.0 | mg |
| Stearic acid | 10.0 | mg |
| Peppermint oil | 6.0 | mg |
| light anhydrous silicic acid | 1.0 | mg |
| talc | 1.0 | mg |
| magnesium stearate | 1.5 | mg |

Formulation 3)

| Suckable pastille | per tablet | |
|---|---|---|
| Ambroxol hydrochloride | 20.0 | mg |
| Noscapine | 5.0 | mg |
| Dequalinium Chloride | 0.125 | mg |
| Sucrose (purified) | 908.675 | mg |
| I-Menthol | 1.0 | mg |
| Peppermint oil | 0.6 | mg |
| Lemon flavour | 3.6 | mg |
| Corn starch | 30.0 | mg |
| Polyvinylpyrrolidone | 21.0 | mg |
| Magnesium Stearate | 10.0 | mg |

Formulation 4)

| Suckable pastille | per tablet | |
|---|---|---|
| Ambroxol hydrochloride | 20.0 | mg |
| Dextromethorphan phenolphthalinate | 10.0 | mg |
| Potassium guaiacolsulphonate | 23.3 | mg |
| Cetylpyridinium Chloride | 1.0 | mg |
| Sucrose (purified) | 869.7 | mg |
| Peppermint flavour | 16.0 | mg |
| Corn starch | 30.0 | mg |
| Polyvinylpyrrolidone | 20.0 | mg |
| Magnesium Stearate | 10.0 | mg |

Formulation 5)

| Suckable pastille | per tablet | |
|---|---|---|
| Ambroxol hydrochloride | 20.0 | mg |
| dl-Methylephedrine Hydrochloride | 6.25 | mg |
| Chlorhexidine Hydrochloride | 5.0 | mg |
| Lactose | 905.25 | mg |
| Low-substituted | 25.0 | mg |
| Hydroxypropylcellulose | | |
| Hydroxypropylcellulose | 20.0 | mg |
| Peppermint flavour | 16.0 | mg |
| Magnesium Stearate | 2.5 | mg |

Formulation 6)

| Suckable pastille | per tablet | |
|---|---|---|
| Ambroxol hydrochloride | 20.0 | mg |
| Ammonium glycyrrhizate | 1.67 | mg |
| Potassium Cresolsulphonate | 30.0 | mg |
| Lactose | 884.83 | mg |
| Low-substituted Hydroxypropylcellulose | 25.0 | mg |
| Hydroxypropylcellulose | 20.0 | mg |
| Peppermint flavour | 16.0 | mg |
| Magnesium Stearate | 2.5 | mg |

The present invention is not to be limited in scope by the specific embodiments described herein, which are intended as single illustrations of individual aspects of the invention, and functionally equivalent methods and components are within the scope of the invention.

Indeed, various modifications of the invention, in addition to those shown and described herein will become apparent to those skilled in the art from the foregoing description and accompanying drawings. Such modifications are intended to fall within the scope of the appended claims.

Various patent applications and publications are cited herein, the disclosures of which are incorporated by reference in their entireties.

What is claimed is:

1. Use of ambroxol or one of the pharmacologically acceptable salts thereof for preparing a pharmaceutical composition for the treatment of pain in the oral and/or pharyngeal cavity, selected from among acute sore throat, aphthae, gingivitis, parodontopathies, pressure points caused by prostheses, pain after oro-pharyngeal interventions, lesions on the mucous membrane in the oral and pharyngeal cavity and herpes simplex in the oral and pharyngeal cavity.

2. Pharmaceutical composition containing ambroxol or one of the pharmacologically acceptable salts thereof and one or more active substances selected from among the antiseptics, vitamins, corticoids, antiphlogistics, antibiotics, antimycotics and proteolytic enzymes.

3. Pharmaceutical composition containing ambroxol or one of the pharmacologically acceptable salts thereof and one or more active substances selected from the group consisting of lysozyme hydrochloride, dipotassium glycyrrhizinate, ammonium glycyrrhizinate, cetylpyridinium chloride, chlorpheniramine maleate, noscapine, dequalinium chloride, dextromethorphane, phenolphthalinate, potassium guaiacolsulphonate, dl-methylephedrine hydrochloride, chlorhexidine hydrochloride, and potassium cresolsulphonate.

4. Pharmaceutical composition consisting of ambroxol, bromhexine or the pharmacologically acceptable salts thereof and pharmaceutical excipients thereof.

5. Pharmaceutical composition according to one of claims 2 to 5, characterised in that the single dose contains 15 to 50 mg ambroxol.

6. Solid, suckable or slowly dissolving formulation of a pharmaceutical composition according to one of claims 2 to 4.

7. Semisolid formulation of a pharmaceutical composition according to one of claims 2 to 5 in the form of a gel.

8. Use of a pharmaceutical composition according to one of claims 2 to 5 for preparing a medicament for treating pain in the oral and/or pharyngeal cavity, selected from among acute sore throat, aphthae, gingivitis, parodontopathies, pressure points caused by prostheses, pain after oro-pharyngeal interventions, lesions on the mucous membrane in the oral and pharyngeal cavity and herpes simplex in the oral and pharyngeal cavity.

9. Use of a pharmaceutical composition consisting of ambroxol hydrochloride, a flavouring, a lubricant, a matrix material, a sweetening agent and a polyethyleneglycol for preparing a medicament for the treatment of pain in the oral and/or pharyngeal cavity, selected from among acute sore throat, aphthae, gingivitis, parodontopathies, pressure points caused by prostheses, pain after oro-pharyngeal interventions, lesions on the mucous membrane in the oral and pharyngeal cavity and herpes simplex in the oral and pharyngeal cavity.

10. Use of a suckable tablet containing ambroxol based on sugar alcohols as the matrix material, characterised in that it contains a pharmaceutically acceptable layered silicate and a polyethyleneglycol, optionally together with other pharmaceutical excipients, taste or flavouring agents for treating pain in the oral and/or pharyngeal cavity, selected from among acute sore throat, aphthae, gingivitis, parodontopathies, pressure points caused by prostheses, pain after oro-pharyngeal interventions, lesions on the mucous membrane in the oral and pharyngeal cavity and herpes simplex in the oral and pharyngeal cavity.

11. Use of ambroxol according to claim 1 for preparing a pharmaceutical composition with a pain-relieving effect lasting for a period of at least 3 hours after administration.

12. Use of a pharmaceutical composition according to one of claims 2 to 5 for preparing a pharmaceutical composition with a pain-relieving effect lasting for a period of at least 3 hours after administration.
